(19) United States
(12) Patent Application Publication
(10) Pub. No.: US 2007/0197488 A1
(43) Pub. Date: Aug. 23, 2007

(54) Prodrugs of ERbeta-selective substances, process for their production, and pharmaceutical compositions that contain these compounds

(76) Inventors: Olaf Peters, Tabarz (DE); Gudrun Reddersen, Jena (DE); Ralf Wyrwa, Oelknitz (DE); Alexander Hillisch, Velbert (DE); Walter Elger, Berlin (DE); Katja Prelle, Berlin (DE); Peter Droescher, Weimar (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/605,472

(22) Filed: Nov. 29, 2006

Related U.S. Application Data
(60) Provisional application No. 60/742,557, filed on Dec. 6, 2005.

(30) Foreign Application Priority Data
Nov. 29, 2005 (DE) 10 2005 05 7225.1

(51) Int. Cl. A61K 31/565

(52) U.S. Cl. 514/171; 552/650

(57) ABSTRACT

This invention provides prodrugs of 8β-substituted estratrienes of general formula (I), in which the group Z is bonded to the steroid, 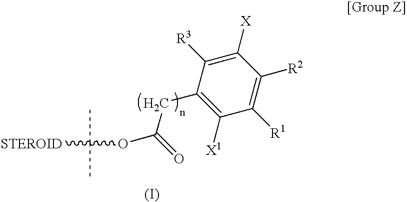 process for their production, pharmaceutical compositions that contain these compounds as well as use thereof. The compounds of general formula I according to the invention do not bind to the estrogen receptor α and/or β. They bind to carbonic anhydrases and inhibit these enzymes.

This application claims the benefit of the filing date of U.S. Provisional Application Ser. No. 60/742,557, filed Dec. 6, 2005.

The invention relates to prodrugs of ERβ-selective substances of general formula (I), 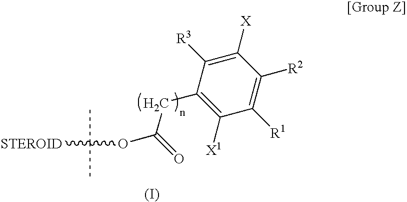
a process for their production, pharmaceutical compositions that contain these compounds, and their use for the production of pharmaceutical agents.

Estrogens play an important role in the organism in both sexes. In the maturing organism, estrogens are involved in the imprinting of sex characteristics. In both sexes, estrogens control the changes in the organism during sexual maturation, such as growth spurts and then the completion of bone growth. In all phases of life, estrogens play a central role (1, 4) in bone metabolism in both sexes. Their loss results in the degradation of bone substance and involves the risk of an elevated brittleness of the bone.

In women, the estrogens that are secreted by the ovary predominate in the organism. In pregnancy, the placenta forms large amounts of estrogen. In men, estrogens are produced primarily “peripherally” by the aromatization of testosterone or the adrenal androgens in various effector organs, such as the central nervous system (CNS), the bones or the intestinal epithelium. This adaptation makes possible physiological estrogen effects in men at very low estradiol levels in the blood. In men and women with a genetic defect of the aromatase or the estrogen receptor, the bones are severely disrupted relative to growth and development (2).

While for natural estrogens, the oral administration (10) is problematic owing to its low oral bioavailability, conventional chemically modified estrogens with improved bioavailability (for example ethinyl estradiol) often have the drawback of producing a considerably increased estrogenic action in the liver (3, 9, 10). This hepatic estrogeneity relates to a number of functions, such as transport proteins, lipometabolism, blood pressure regulation and clotting factors (5, 7, 11, 12, 14). Also, the especially important secretion of IGF-I (8) for the preservation of muscles and bones is negatively affected by hepatic estrogenic actions (12, 13, 6).

In WO 01/77139, new 8β-substituted estratrienes are described, whereby the 8β-substituent can be a straight-chain or branched-chain, optionally partially or completely halogenated alkyl or alkenyl radical with up to 5 carbon atoms, an ethinyl or a prop-1-inyl radical, which as pharmaceutical active ingredients show a higher in vitro affinity to estrogen receptor preparations of rat prostates than to estrogen receptor preparations of rat uteri and have in vivo a preferential action on bone in comparison to the uterus and/or a pronounced action with respect to the stimulation of the expression of 5HT2a-receptors and -transporters. These compounds can preferably be used for treating diseases that are caused by an estrogen deficiency.

Drawbacks of these 8β-substituted estratrienes are their deficient oral bioavailability as well as the metabolic instability.

From WO 01/91797, steroidal compounds are known that are bonded via a group —SO₂NR¹R² to erythrocytes and accumulate there. The concentration ratio of the compounds between erythrocytes and plasma is 10-1000:1, preferably 30-1000:1, such that a depot formation in the erythrocytes can be mentioned. By the strong bond of the compounds to the erythrocytes, the metabolization is avoided during the liver passage. Disadvantageously, despite a reduced metabolization with the indicated dosages, no therapy-relevant active ingredient levels are given.

It is therefore the object of this invention to provide prodrugs of ERβ-selective compounds, which make the ERβ-selective compounds orally bioavailable.

This object is achieved by sulfamoyl compounds of 8β-substituted estratrienes of general formula (I), in which group Z is bonded to the steroid that is to be released

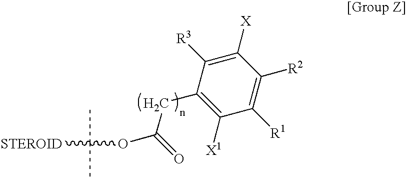

in which n can mean a number 0-4,

R¹ means a radical —SO₂NH₂ or —NHSO₂NH₂,
whereby R², R³ and X, X¹, independently of one another, stand for a hydrogen atom, a halogen atom, a nitrile group, a nitro group, a C₁₋₅-alkyl group, a CₚF₂ₚ₊₁ group with p=1-3, a group OC(O)—R²⁰, COOR²⁰, OR²⁰, C(O)NHR²⁰ or OC(O)NH—R²¹,
whereby R²⁰ and R²¹ are a C₁₋₅-alkyl group, a C₃₋₈-cycloalkyl group, an aryl group, a C₁₋₄-alkylene aryl group, a C₁₋₄-alkylene-C₃₋₈-cycloalkyl group or a C₃₋₈-cycloalkylene-C₁₋₄-alkyl group, and
R²⁰ in addition can mean a hydrogen atom, or

R² can mean a radical —SO₂NH₂ or —NHSO₂NH₂,
whereby R¹, R³ and X, X¹, independently of one another, stand for a hydrogen atom, a halogen atom, a nitrile group, a nitro group, a C₁₋₅-alkyl group, a CₚF₂ₚ₊₁ group with p=1-3, a group OC(O)—R²⁰, COOR²⁰, OR²⁰, C(O)NHR²⁰ or OC(O)NH—R²¹,
whereby R²⁰ and R²¹ are a C₁₋₅-alkyl group, a C₃₋₈-cycloalkyl group, an aryl group, a C₁₋₄-alkylene aryl group, a C₁₋₄-alkylene-C₃₋₈-cycloalkyl group or C₃₋₈-cycloalkylene-C₁₋₄-alkyl group, and
R²⁰ in addition can mean a hydrogen, or

R³ can mean a radical —SO₂NH₂ or —NHSO₂NH₂,
whereby R¹, R² and X, X¹, independently of one another, stand for a hydrogen atom, a halogen atom, a nitrile group, a nitro group, a C₁₋₅-alkyl group, a CₚF₂ₚ₊₁ group with p=1-3, a group OC(O)—R²⁰, COOR²⁰, OR²⁰, C(O)NHR²⁰ or OC(O)NH—R²¹,
whereby R²⁰ and R²¹ are a C₁₋₅-alkyl group, a C₃₋₈-cycloalkyl group, an aryl group, a C₁₋₄-alkylene aryl group, a C₁₋₄-alkylene-C₃₋₈-cycloalkyl group or C₃₋₈-cycloalkylene-C₁₋₄-alkyl group, and
R²⁰ in addition can mean a hydrogen, and

STEROID stands for a steroidal ABCD-ring system of formula (A):

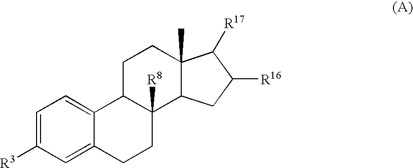

whereby the radicals R³, R⁸, R¹⁶ and R¹⁷ have the following meaning:
R³ can be Z and
R¹⁷ can be an OH group, a tri(C₁-C₄-alkyl)silyloxy group or a group OC(O)—R²⁰ or
R³ can be OH, OMe, a tri(C₁-C₄-alkyl)silyloxy group, or a group OC(O)—R²⁰ and
R¹⁷ can be Z
and
R⁸ can be a branched or straight-chain, optionally partially or completely halogenated alkyl, alkenyl or alkinyl radical with up to 3 carbon atoms,
R¹⁶ can be a hydrogen atom, a halogen atom, or a methyl group,

whereby the substituents R¹⁶ and R¹⁷ in each case can be both in α- and in β-position, and their pharmaceutically acceptable salts.

In addition, this invention comprises the new compounds as pharmaceutical active ingredients, their production, their therapeutic application and pharmaceutical dispensing forms that contain the new substances.

The invention relates to estrogen derivatives that themselves cannot bind to the estrogen receptor and from which the contained mother estrogen is released in the body, process for their production, and pharmaceutical compositions that contain these compounds. The compounds according to the invention are prodrugs that release an ERβ-selective estrogen (mother estrogen) after saponification of the ester group Z.

By absolute and relatively greatly weakened action via the ER α, undesirable estrogen effects of any standard estrogen therapy on the uterus, the mammary glands and the liver are avoided, as they are typical for non-dissociated estrogens. The compounds according to the invention have therapeutically advantageous estrogenic activities if they are mediated by the ER β, in particular in the central nervous system, in the circulatory system and in the bones.

The substances according to the invention are preferably used for oral therapy. Compared to their mother estrogens, the compounds according to the invention have a clearly increased oral bioavailability, an increased systemic, but generally reduced hepatic estrogeneity. By this dissociation of desirable and undesirable hormonal effects, simultaneously more therapeutically effective and, in comparison to the prior art, more compatible pharmaceutical agents are made possible.

The substances according to the invention are cleaved enzymatically or hydrolytically in the body, whereby no steroid sulfatases (STS), such as, for example, for cleavage of estradiol-3-sulfamate, are required. Thus, the inhibition of the steroid sulfatase that is typical for estrogen-3-sulfamates and disadvantageous for achieving strong estrogenic effects can also be avoided, which is typical for estrogen sulfamates in humans. In the case of oral therapy with natural estrogens (estradiol, estradiol valerate, estrone sulfate, conjugated estrogens), but also in that with estradiol sulfamate, high levels of estrone dominate in the blood (10). Unlike in the cycle, the concentrations of estradiol in the blood are lower than that of estrone. This is therefore disadvantageous, since estrone is a less effective estrogen than estradiol.

An advantage of the substances according to the invention in comparison to those in the prior art is the preferable release of the respective mother estrogen, thus, for example, 8β-ethylestradiol, 8β-methylestradiol, 8B-vinylestradiol and 8B-difluorovinylestradiol instead of the inactive estrone derivatives.

The compounds of general formula (I) according to the invention or their pharmaceutically acceptable salts can be used as individual components in pharmaceutical preparations or in combination in particular with antiestrogens or gestagens. The combination with ERα-selective antiestrogens or with antiestrogens that are peripherally-selectively active, i.e., that do not pass through the blood-brain barrier, is especially preferred.

The substances and the pharmaceutical agents containing them are especially suitable for the treatment of perimenopausal and postmenopausal symptoms, in particular hot flashes, sleep disorders, irritability, mood swings, incontinence, vaginal atrophy, and hormone-deficiency-induced mental disorders. The substances are also suitable for hormone substitution and for the treatment of hormone-deficiency-induced symptoms in ovarian dysfunction that is caused by surgery, medication, etc. Prevention of bone mass loss in postmenopausal women and andropausal men, in women who have undergone hysterectomies or in women who were treated with LHRH antagonists or agonists is also part of this.

The prodrugs of ERβ-selective agonists according to the invention can be used alone or in combination with antiestrogens, aromatase inhibitors or selective estrogen receptor modulators (SERM) for the treatment of prostate hyperplasia to avoid estrogen deprivation or to reduce the effects thereof.

As antiestrogen, preferably 7alpha-[9-[(4,4,5,5,5-pentafluoropentyl)sulfinyl]-nonyl]estra-1,3,5(10)-triene-3,17β-diol (fulvestrant) is used.

As the aromatase inhibitors that are to be used, the following are considered: anastrozole, atamestane, fadrozole, formestane, and letrozole.

As SERM, compounds that are selected from the following group are considered: raloxifene, tamoxifen, and 5-(4-{5-[(RS)-(4,4,5,5,5-pentafluoropentyl)sulfinyl]-pentyl}phenyl)-6-phenyl-8,9-dihydro-7H-benzocyclohepten-2-ol (WO 00/03979).

The compounds are also suitable for alleviating symptoms of andropause and menopause, i.e., for male and female hormone replacement therapy (HRT), namely both for prophylaxis and for treatment, in addition for treating symptoms accompanied by a dysmenorrhea as well as for treating acne.

In addition, the substances can be used for prophylaxis against hormone-deficiency-induced bone mass loss and osteoporosis, for preventing cardiovascular diseases, in particular vascular diseases such as arteriosclerosis, for inhibiting the proliferation of arterial smooth muscle cells, and for treating primary pulmonary high blood pressure.

In addition, the substances can be used for treating inflammatory diseases and diseases of the immune system, in particular autoimmune diseases, such as, e.g., rheumatoid arthritis, multiple sclerosis, Crohn's disease as well as endometriosis.

The compounds can be used in particular according to therapies that result in estrogen deprivation, for example after treatment with aromatase inhibitors or GnRH antagonists or agonists, for treatment of arthritic symptoms.

In addition, the compounds can be used for treatment of male fertility disorders and prostatic diseases. The compounds according to the invention are suitable for estrogen treatment of prostate cancer.

The compounds can also be used in combination with the natural vitamin D3 or with calcitriol analogs for bone building or as supporting therapies to therapies that cause a bone mass loss (for example, a therapy with glucocorticoids, aromatase inhibitors, GnRH agonists or antagonists (chemotherapy)).

Finally, the compounds of general formula (I) in connection with progesterone receptor modulators, for example mesoprogestins, such as asoprisnil, can be used, specifically especially for use in hormone replacement therapy and for treatment of gynecological disorders.

In addition, the compounds of general formula (I) according to the invention can be used for the treatment of alopecia, caused by, for example, chemotherapy.

A therapeutic product that contains an estrogen and a pure antiestrogen for simultaneous, sequential or separate use for the selective estrogen therapy of perimenopausal or postmenopausal conditions is already described in EP-A 0 346 014.

In terms of this invention, “C₁₋₅-alkyl group” is defined as a branched or straight-chain alkyl radical with up to 5 carbon atoms, which can be substituted by, for example, halogens, OH, or CN. As examples, methyl, ethyl, n-propyl, i-propyl, n-butyl, i-butyl, tert-butyl or n-pentyl can be mentioned.

The above-mentioned “C₃₋₈-cycloalkyl group” according to the invention means a monocyclic or bicyclic group, which can be substituted by, for example, halogens such as fluorine, chlorine or bromine, OH or CN, such as, for example, a cyclopropyl, cyclobutyl, cyclopentyl, cyclohexyl or a hydroxycyclohexyl group.

In terms of this application, the term “aryl group” is defined as a substituted or unsubstituted aryl radical with 6 to 15 carbon atoms, for example a phenyl group, a substituted phenyl group, such as a halophenyl group, or a nitrophenyl group, or a naphthyl group.

In terms of this application, the term “C₁₋₄-alkylene aryl group” is defined as a di-substituted alkyl radical, which is substituted with at least one aryl radical. Both radicals together have 7 to 15 carbon atoms, whereby the aryl radical can carry additional substituents, such as, for example, a halogen atom. Examples are a benzyl group or a halobenzyl group.

In terms of this application, the term “C₁₋₄-alkylene-C₃₋₈-cycloalkyl group” is defined as a di-substituted alkyl radical that is substituted with a C₃₋₈-cycloalkyl radical. Both radicals together have 4 to 12 carbon atoms, whereby the cycloalkyl radical can carry additional substituents, such as, for example, a halogen atom. Examples are a cyclopentylethyl, cyclohexylmethyl or cyclohexylethyl group.

In terms of this application, the term “C₃₋₈-cycloalkylene-C₁₋₄-alkyl group” is defined as a di-substituted C₃₋₈-cycloalkylene radical that is substituted with a C₁₋₄-alkyl radical. Both radicals together have 4 to 12 carbon atoms, whereby the group can carry additional substituents, such as, for example, a halogen atom. Examples are a propylcyclohexyl or butylcyclohexyl group.

A trialkylsilyloxy group is, for example, a trimethylsilyloxy group or a tert-butyldimethylsilyloxy group.

Within the scope of this invention, the term “halogen atom” is defined as a fluorine, chlorine, bromine or iodine atom. Fluorine, chlorine and bromine are preferred.

The number “n” is preferably 0, 1 or 2.

R¹ preferably means a group —SO₂NH₂, whereby R², R³, X¹ and X, independently of one another, are preferably an H, F, or Cl atom, or an OH or a methoxy group.

R² preferably means a group —SO₂NH₂, whereby R¹, R³, X¹ and X, independently of one another, are preferably an H, F, or Cl atom, or an OH or a methoxy group.

R³ preferably means a group —SO₂NH₂, whereby R¹, R², X¹ and X, independently of one another, preferably are an H, F, or Cl atom, or an OH or a methoxy group.

X¹ is preferably an H atom.

R⁸ is preferably methyl, ethyl, vinyl, difluorovinyl, ethinyl or prop-1-inyl.

Methyl, ethyl, vinyl or difluorovinyl are especially preferred for R⁸.

Y is preferably OH, OMe, a trimethylsilyloxy, a tert-butyldimethylsilyloxy, a benzoate, a sulfamoyl benzoate, acetate, propionate, valerate, butcyclate or cyclopentylpropionate radical.

R¹⁷ preferably means an OH, a trimethylsilyloxy, an acetate, propionate, valerate, a benzoate, or an optionally halogenated sulfamoyl benzoate radical.

In terms of this invention, especially preferred compounds are cited below:
1) (3′-Hydroxy-8′β-methyl-estra-1′,3′,5′(10′)-trien-17′β-yl)3-sulfamoyl benzoate
2) (3′-Hydroxy-8′β-ethyl-estra-1′,3′,5′(10′)-trien-17′β-yl)3-sulfamoyl benzoate,
3) (3′-Hydroxy-8′β-vinyl-estra-1′,3′,5′(10′)-trien-17′β-yl)3-sulfamoyl benzoate,
4) (3′-Hydroxy-8′β-methyl-estra-1′,3′,5′(10′)-trien-17′β-yl)4-sulfamoyl benzoate,
5) (3′-Hydroxy-8′β-ethyl-estra-1′,3′,5′(10′)-trien-17′β-yl)4-sulfamoyl benzoate,
6) (3′-Hydroxy-8′β-vinyl-estra-1′,3′,5′(10′)-trien-17′β-yl)4-sulfamoyl benzoate,
7) (3′-Acetoxy-8′β-methyl-estra-1′,3′,5′(10′)-trien-17′β-yl)3-sulfamoyl benzoate,
8) (3′-Acetoxy-8′β-ethyl-estra-1′,3′,5′(10′)-trien-17′β-yl)3-sulfamoyl benzoate
9) (3′-Acetoxy-8′β-vinyl-estra-1′,3′,5′(10′)-trien-17′β-yl)3-sulfamoyl benzoate
10) (3′-Acetoxy-8′β-methyl-estra-1′,3′,5′(10′)-trien-17′β-yl)4-sulfamoyl benzoate,
11) (3′-Acetoxy-8′β-ethyl-estra-1′,3′,5′(10′)-trien-17′β-yl)4-sulfamoyl
12) (3′-Acetoxy-8′β-vinyl-estra-1′,3′,5′(10′)-trien-17′β-yl)4-sulfamoyl benzoate
13) (3′-Benzoyloxy-8′β-methyl-estra-1′,3′,5′(10′)-trien-17′β-yl)3-sulfamoyl benzoate,
14) (3′-Benzoyloxy-8′β-methyl-estra-1′,3′,5′(10′)-trien-17′β-yl)4-sulfamoyl benzoate,
15) (3′-Benzoyloxy-8′β-ethyl-estra-1′,3′,5′(10′)-trien-17′β-yl)3-sulfamoyl benzoate,
16) (3′-Benzoyloxy-8′β-ethyl-estra-1′,3′,5′(10′)-trien-17′β-yl)4-sulfamoyl benzoate,
17) (3′-Benzoyloxy-8′β-vinyl-estra-1′,3′,5′(10′)-trien-17′β-yl)3-sulfamoyl benzoate,
18) (3′-Benzoyloxy-8′β-vinyl-estra-1′,3′,5′(10′)-trien-17′β-yl)4-sulfamoyl benzoate,
19) (3′-Hydroxy-8′β-vinyl-estra-1′,3′,5′(10′)-trien-17′β-yl)2-chloro-5-sulfamoyl benzoate,
20) (3′-Hydroxy-8′β-vinyl-estra-1′,3′,5′(10′)-trien-17′β-yl)3-sulfamoyl-4-chloro-benzoate,
21) (3′-Hydroxy-8′β-methyl-estra-1′,3′,5′(10′)-trien-17′β-yl)2-chloro-5-sulfamoyl benzoate,
22) (3′-Hydroxy-8′β-methyl-estra-1′,3′,5′(10′)-trien-17′β-yl)3-sulfamoyl-4-chlorobenzoate,
23) (3′-Hydroxy-8′β-ethyl-estra-1′,3′,5′(10′)-trien-17′β-yl)2-chloro-5-sulfamoyl benzoate,
24) (3′-Hydroxy-8′β-ethyl-estra-1′,3′,5′(10′)-trien-17′β-yl)3-sulfamoyl-4-chloro-benzoate,
25) (17′β-(n-Pentanoyloxy)-8′β-vinyl-estra-1′,3′,5′(10′)-trien-3′-yl)3-sulfamoyl benzoate,
26) (17′β-(n-Pentanoyloxy)-8′β-methyl-estra-1′,3′,5′(10′)-trien-3′-yl)3-sulfamoyl benzoate,
27) (17′β-(n-Pentanoyloxy)-8′β-ethyl-estra-1′,3′,5′(10′)-trien-3′-yl)3-sulfamoyl benzoate,
28) (17′β-Benzoyloxy-8′β-vinyl-estra-1 ′,3′,5′(10′)-trien-3′-yl)3-sulfamoyl benzoate,
29) (17′β-Benzoyloxy-8′β-methyl-estra-1′,3′,5′(10′)-trien-3′-yl)3-sulfamoyl benzoate,
30) (17′β-Benzoyloxy-8′β-ethyl-estra-1′,3′,5′(10′)-trien-3′-yl)3-sulfamoyl benzoate,
31) (17′β-(n-Pentanoyloxy)-8′β-vinyl-estra-1′,3′,5′(10′)-trien-3′-yl)4-sulfamoyl benzoate,
32) (17′β-(n-Pentanoyloxy)-8′β-methyl-estra-1′,3′,5′(10′)-trien-3′-yl)4-sulfamoyl benzoate,
33) (17′β-(n-Pentanoyloxy)-8′β-ethyl-estra-1′,3′,5′(10′)-trien-3′-yl)4-sulfamoyl benzoate,
34) (17′β-Benzoyloxy-8′β-vinyl-estra-1′,3′,5′(10′)-trien-3′-yl)4-sulfamoyl benzoate,
35) (17′β-Benzoyloxy-8′β-methyl-estra-1′,3′,5′(10′)-trien-3′-yl)4-sulfamoyl benzoate,
36) (17′β-Benzoyloxy-8′β-ethyl-estra-1′,3′,5′(10′)-trien-3′-yl)4-sulfamoyl benzoate,
37) (17′β-Acetoxy-8′β-vinyl-estra-1′,3′,5′(10′)-trien-3′-yl)3-sulfamoyl benzoate,
38) (17′β-Acetoxy-8′β-ethyl-estra-1′,3′,5′(10′)-trien-3′-yl)3-sulfamoyl benzoate,
39) (17′β-Acetoxy-8′β-methyl-estra-1′,3′,5′(10′)-trien-3′-yl)3-sulfamoyl benzoate,
40) (17′β-Acetoxy-8′β-vinyl-estra-1′,3′,5′(10′)-trien-3′-yl)4-sulfamoyl benzoate,
41) (17′β-Acetoxy-8′β-ethyl-estra-1′,3′,5′(10′)-trien-3′-yl)4-sulfamoyl benzoate
42) (17′β-Acetoxy-8′β-methyl-estra-1′,3′,5′(10′)-trien-3′-yl)4-sulfamoyl benzoate,
43) (3′-Methoxy-8′β-vinyl-estra-1′,3′,5′(10′)-trien-17′β-yl)3-sulfamoyl benzoate,
44) (3′-Methoxy-8′β-ethyl-estra-1′,3′,5′(10′)-trien-17′β-yl)3-sulfamoyl benzoate
45) (3′-Methoxy-8′β-methyl-estra-1′,3′,5′(10′)-trien-17′β-yl)3-sulfamoyl benzoate,
46) (3′-Methoxy-8′β-vinyl-estra-1′,3′,5′(10′)-trien-17′β-yl)4-sulfamoyl benzoate,
47) (3′-Methoxy-8′β-ethyl-estra-1′,3′,5′(10′)-trien-17′β-yl)4-sulfamoyl benzoate
48) (3′-Methoxy-8′β-methyl-estra-1′,3′,5′(10′)-trien-17′β-yl)4-sulfamoyl benzoate.
   In-Vivo Tests
   Principle of the Test and Test Description:

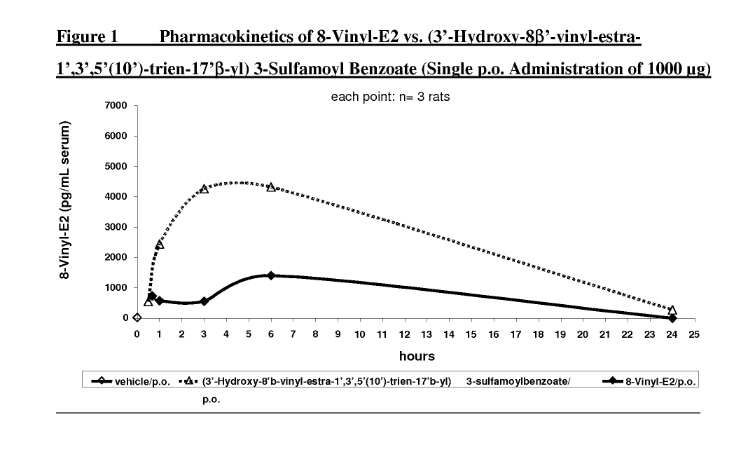

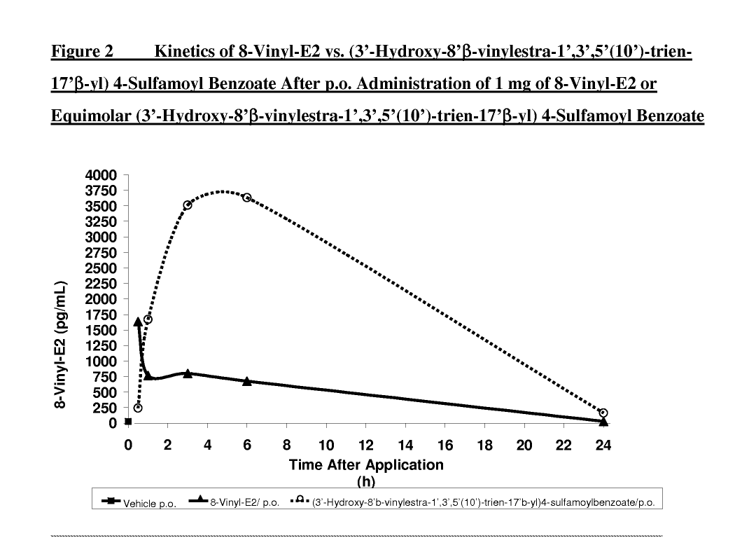

Adult Wistar rats were ovariectomized 14 days after this operation for the study of the substances according to the invention. A treatment extended over 3 days (days 1-3), and on day 4, the animals were sacrificed. Then, the recovery of plasma for hormone-analytical and clinical-chemical determinations and the determination of uterus weights were carried out. In satellite tests, correspondingly conditioned animals were sacrificed and samples were taken of their blood after one-time treatment and at other times (see FIGS. 1 and 2).

Increase of the 8β-vinylestra-1,3,5(10)-triene-3,17β-diol level (8-vinyl-E2) in the plasma of rats after 1× oral administration of 1 mg/animal. Considerably greater increase of the 8-vinyl-E2 level after administration of 1 mg/animal of (3′-hydroxy-8β′-vinyl-estra-1′,3′,5′(10′)-trien-17′β-yl)3-sulfamoyl benzoate than after oral administration of 8-vinyl-E2.

Increase of the 8β-vinylestra-1,3,5(10)-triene-3,17β-diol level (8-vinyl-E2) in the plasma of rats after a one-time oral administration of 1 mg/animal. A greater increase of the 8-vinyl-E2 level can be observed clearly after the administration of 1 mg/animal of (3′-hydroxy-8′β-vinylestra-1′,3′,5′(10′)-trien-17′β-yl)4-sulfamoyl benzoate than after oral administration of 8-vinyl-E2.

In in vivo experiments in rats, it was found that after oral administration of the compounds according to the invention, an unexpectedly high increase of the mother estrogen can be noted. This is not the case, for example, in the 17-benzoates and 17-acetates of 8β-vinyl-estradiol.

Unlike conventional estrogens, the substances according to the invention do not have any action on the uterus, the ovary and the liver.

In-Vitro Tests

a) Blood Plasma Concentration Ratio—Test Principle and Test Description:

The SO₂—NH₂ group of the substances according to the invention can lead to a concentration in erythrocytes by binding to carbonic anhydrases. The displacement of estradiol-3-sulfamate from the erythrocyte bond is measured by test substances.

Test preparation: Human blood is mixed with a mixture that consists of ¹⁴C-labeled and unlabeled estradiol sulfamate. At the selected working point, the erythrocytes are saturated, and the distribution in plasma/erythrocytes is 40:60. A second blood sample is mixed with a mixture that consists of ¹⁴C-labeled etradiol sulfamate and unlabeled test substance. The relative binding affinity is calculated from the portion of ¹⁴C-labeled estradiol sulfamate in the plasma: high proportion=strong displacement of ¹⁴C-estradiol sulfamate from the erythrocytes by the test substance=high binding affinity.

In contrast to the results published in WO 01/91797, the concentration ratios of the compounds according to the invention between erythrocytes and plasma do not lie in a range of 10-1000:1, but rather in a range <10:1. In the case of (3′-hydroxy-8β′-vinyl-estra-1′,3′,5′(10′)-trien-17′β-yl)3-sulfamoyl benzoate, the ratio is, for example, approximately 1.4:1.

b) Carbonic Anhydrase Inhibition—Test Principle and Test Description:

Carbonic anhydrases catalyze the CO₂ hydration.

Test preparation: A constant CO₂ stream is directed through a buffer that was mixed with carbonic anhydrase I or carbonic anhydrase II. The measuring parameter is the time that is required to drop the pH within defined limits. This parameter reflects the formation of H₂CO₃ in the medium. IC₅₀-Inhibiting values are determined by test substances being pipetted into the test preparation. In the concentration areas that are examined, the test substances cause no to complete inhibition of the above-mentioned enzymes.

TABLE 1
IC₅₀-Inhibiting Values of Human Carbonic Anhydrases I and II

| | CAI | | CAII | |
|---|---|---|---|---|
| | | IC₅₀ (nM) | | IC₅₀ (nM) |
| Inhibitor | IC₅₀ (nM) | Literature | IC₅₀ (nM) | Literature |
| Estradiol-3-sulfamate | 157 ± 10.6 | — | 21.6 ± 1.5 | — |
| (3′-Hydroxy-8′beta-vinyl- | 3900 | — | 570 | — |
| estra-1′,3′,5′(10′)-trien- | | | | |
| 17′beta-yl)3- | | | | |
| sulfamoylbenzoat | | | | |
| (3′-Hydroxy-8′beta-vinyl- | >10000 | — | >10000 | — |
| estra-1′,3′,5′(10′)-trien- | | | | |
| 17′beta-yl)-benzoat | | | | |
| Acetazolamid | 1200 | 1900 | 60 | 90¹ |
| (bekannter CA-Hemmer) | | | | |

¹Literature: C. Landolfi, M. Marchetti, G. Ciocci, and C. Milanese, Journal of Pharmacological and Toxicological Methods 38, 169-172 (1997).
[Key to Table 1:
-benzoat = -benzoate
Acetazolamid (bekannter CA-Hemmer) = Acetazolamide (of known CA inhibitors)

Despite the low blood-plasma concentration ratios, a binding (inhibition) to the two isoenzymes carbonic anhydrases CA I and CA II in the erythrocytes could be shown in all cases. The binding to erythrocytes induced by affinity to the carbonic anhydrases is important for the properties as estrogen. This binding is essential for a reduced extraction of the orally administered substance in the first liver passage. High or low affinity to the erythrocytic carbonic anhydrases, faster or delayed release from this depot and subsequent hydrolysis determine the therapeutic applicability of the substances according to the invention. The compounds according to the invention thus open up the possibility of achieving higher, shorter-term or uniformly low and longer-lasting hormone levels with an equimolar amount of substance administered. As a result, active strength and duration of action are varied and make possible a therapy adapted to the organism.

These test results open up many possible applications in the compounds of general formula (I) according to the invention for hormone replacement therapy (HRT) and in hormonally-induced diseases in men and women.

Subjects of this invention are therefore also pharmaceutical compositions that contain at least one compound of general formula (I), optionally together with pharmaceutically compatible adjuvants and vehicles.

Compared to their mother estrogens, the substances according to the invention have pharmacologically and pharmacokinetically improved properties that are based on a reduced hepatic extraction and more uniform and longer-lasting blood levels of the released estrogen.

Dosage

The Erβ-selective compounds of general formula (I) are administered orally for use according to the invention.

Suitable dosages of the compounds according to the invention in humans for the treatment of perimenopausal and postmenopausal symptoms, hormone-deficiency-induced symptoms, gynecological disorders such as ovarian dysfunction and endometriosis, male and female fertility disorders, hormone-induced tumor diseases as well as for the use in male and female hormone replacement therapy are, depending on indication, 5 μg to 2000 mg per day, depending on age and constitution of the patient, whereby the necessary daily dose can be administered one or more times.

For gynecological disorders such as ovarian dysfunction and endometriosis, in this case dosages of between 0.5 and 100 mg are considered; for the treatment of male and female fertility disorders, dosages of 5 μg to 50 mg are considered; for hormone-induced tumor diseases, dosages of 5 to 500 mg are considered, and for male or female hormone replacement therapy, dosages of 5 μg to 100 mg are considered.

In addition to commonly used vehicles and/or diluents, the pharmaceutical compositions contain at least one compound of general formula I. The substances according to the invention can also be used therapeutically in combination with a gestagen, antigestagen or mesoprogestin. The substances according to the invention are preferably used individually as active ingredients in pharmaceutical preparations.

The pharmaceutical agents of the invention are produced in a known way with the commonly used solid or liquid vehicles or diluents and the commonly used pharmaceutical-technical adjuvants corresponding to the desired type of administration with a suitable dosage. The preferred preparations exist in a form for dispensing that is suitable for oral administration. Such forms for dispensing are, for example, tablets, film tablets, coated tablets, capsules, pills, powders, solutions or suspensions or else depot forms.

Corresponding tablets can be obtained by, for example, mixing active ingredient with known adjuvants, for example inert diluents such as dextrose, sugar, sorbitol, mannitol, polyvinylpyrrolidone, explosives such as corn starch or alginic acid, binders such as starch or gelatins, lubricants such as magnesium stearate or talc and/or agents for achieving a depot effect, such as carboxylpolymethylene, carboxyl methyl cellulose, cellulose acetate phthalate or polyvinyl acetate. The tablets can also consist of several layers.

Coating cores, which are produced analogously to the tablets, with agents that are commonly used in tablet coatings, for example polyvinyl pyrrolidone or shellac, gum Arabic, talc, titanium oxide or sugar, can accordingly produce coated tablets. In this case, the shell of the coated tablet can also consist of several layers, whereby the adjuvants that are mentioned above in the tablets can be used.

Solutions or suspensions with the compounds of general formula I according to the invention can contain additional taste-improving agents such as saccharine, cyclamate or sugar, as well as, e.g., flavoring substances such as vanilla or orange extract. In addition, they can contain suspending adjuvants such as sodium carboxy methyl cellulose or preservatives, such as p-hydroxybenzoates.

The capsules that contain compounds of general formula I can be produced, for example, by the compound(s) of general formula I being mixed with an inert vehicle such as lactose or sorbitol and encapsulated in gelatin capsules.

Suitable suppositories can be produced by, for example, mixing with vehicles that are provided for this purpose, such as neutral fats or polyethylene glycol or derivatives thereof.

The examples below explain this invention without limiting it.

### EXAMPLE 1

### (3′-Hydroxy-8′-vinylestra-1′,3′,5′(10′)-trien-17′β-yl)-3-sulfamoyl benzoate

### 3,17β-Bis-(tert-butyldimethylsilyloxy)-8-vinyl-estra-1,3,5(10)-triene

1.0 g of 8-vinyl-estra-1,3,5(10)-triene-3,17β-diol was added in 18 ml of DMF and mixed with 2.8 g of imidazole and 3.6 g of tert-butyldimethylchlorosilane. The solution was stirred for 2 hours at room temperature and then extracted with n-hexane. The organic phase was washed with saturated aqueous common salt solution and water, dried on sodium sulfate, and concentrated by evaporation in a vacuum. In this way, 2.0 g of crude 3,17β-bis-(tert-butyldimethylsilyloxy)-8-vinyl-estra-1,3,5(10)-triene is obtained.

¹H-NMR (CDCl₃): 0.01, 0.03 (s, 3H, SiMe₂t-Bu), 0.17 (s, 6H, SiMe₂t-Bu), 0.73 (s, 3H, H-18), 0.88 (s, 9H, SiMe₂t-Bu), 0.96 (s, 9H, SiMe₂t-Bu), 3.54 (t, 1H, H-17), 6.49 (d, 1H, H-4), 6.58 (dd, 1H, H-2), 7.08 (d, 1H, H-1).

### 3-(tert-Butyldimethylsilyloxy)-8-vinylestra-1,3,5(10)-trien-17β-ol

Variant 1

3.79 g of crude 3,17β-bis-(tert-butyldimethylsilyloxy)-8-vinyl-estra-1,3,5(10)-triene from the last stage was dissolved at room temperature in 245 ml of THF and 145 ml of acetonitrile. Then, a solution that consists of 240 ml of acetonitrile, 0.4 ml of water, and 1.2 ml of chlorotrimethylsilane was produced, and 140 ml from this solution was added in drops to the steroid solution. After 21 hours, it was mixed with methylene chloride, washed with water, dried on sodium suflate and concentrated by evaporation in a vacuum. The thus obtained 2.84 g of crude product was purified by column chromatography on silica gel (cyclohexane/ethyl acetate 8:2). In this way, 640 mg (22%) of 3-(tert-butyldimethyl-silyloxy)-8-vinylestra-1,3,5(10)-trien-17β-ol was obtained.

¹H-NMR (CDCl₃): 0.17 (s, 6H, SiMe₂t-Bu), 0.78 (s, 3H, H-18), 0.96 (s, 9H, SiMe₂t-Bu), 3.63 (t, 1H, H-17), 6.49 (d, 1H, H-4), 6.58 (dd, 1H, H-2), 7.08 (d, 1H, H-1).

Variant 2

100 mg of 3,17β-bis-(tert-butyldimethylsilyloxy)-8-vinyl-estra-1,3,5(10)-triene was dissolved in 30 ml of acetone, mixed with 3.5 ml of 5% hydrochloric acid and stirred for 2 hours at room temperature. Then, it was mixed with water and ethyl acetate, the organic phase was separated, washed with water, dried on sodium sulfate and concentrated by evaporation in a rotary evaporator. After chromatographic purification of the crude product on silica gel (cyclohexane/ethyl acetate 9:1), 31 mg (40%) of colorless 3-(tert-butyldimethylsilyloxy)-8-vinylestra-1,3,5(10)-trien-17β-ol was obtained.

### (3′-(tert-Butyldimethylsilyloxy)-8′-vinylestra-1′,3′,5′(10′)-trien-17′β-yl)-3-chlorosulfonylbenzoate

300 mg of 3-(tert-butyldimethylsilyloxy)-8-vinylestra-1,3,5(10)-trien-17β-ol was dissolved in 15 ml of tetrahydrofuran (THF) and mixed with 150 mg of sodium hydride. Then, a solution that consists of 0.3 ml of 3-(chlorosulfonyl)-benzoyl chloride in 3 ml of THF was added in drops and refluxed for 4 hours. The cooled reaction solution was poured onto ice water, extracted with methylene chloride, the organic phase was dried on sodium sulfate and concentrated by evaporation in a rotary evaporator. After column-chromatographic purification on silica gel (cyclohexane), 198 mg (44%) of (3′-(tert-butyldimethylsilyloxy)-8′-vinylestra-1′,3′,5′(10′)-trien-17′β-yl)-3-chlorosulfonylbenzoate was obtained in this way.

### (3′-(tert-Butyldimethylsilyloxy)-8′-vinylestra-1′,3′,5′(10′)-trien-17′β-yl)-3-sulfamoyl benzoate

198 mg of (3′-(tert-butyldimethylsilyloxy)-8′-vinylestra-1′,3′,5′(10′)-trien-17′β-yl)-3-chlorosulfonylbenzoate was mixed with 20 ml of methylene chloride and 20 ml of 25% aqueous ammonia solution and stirred at room temperature. After 2 hours, it was mixed with water and methylene chloride, the phases were separated, and the organic phase was washed neutral with water. After drying on sodium sulfate, it was concentrated by evaporation in a rotary evaporator. Thus, 144 mg (75%) of (3′-(tert-butyldimethylsilyloxy)-8′-vinylestra-1′,3′,5′(10′)-trien-17′β-yl)-3-sulfamoyl benzoate was obtained.

¹H-NMR (CDCl₃): 0.15 (s, 6H, SiMe₂t-Bu), 0.93 (s, 9H, SiMe₂t-Bu), 0.93 (s, 3H, H-18), 4.83 (t, 1H, H-17), 6.47 (d, 1H, H-4), 6.56 (dd, 1H, H-2), 7.09 (d, 1H, H-1).

### (3′-Hydroxy-8′-vinylestra-1′,3′,5′(10′)-trien-17′β-yl)-3-sulfamoyl benzoate

90 mg of tetrabutylammonium fluoride was added to 144 mg of (3′-(tert-butyldimethylsilyloxy)-8′-vinylestra-1′,3′,5′(10′)-trien-17′β-yl)-3-sulfamoyl benzoate ml of tetrahydrofuran, stirred for 2 hours at room temperature, and then mixed with water and methylene chloride. The organic phase was washed neutral with water, dried on sodium sulfate, and concentrated by evaporation in a rotary evaporator. The foamy crude product was purified by column chromatography on silica gel (cyclohexane/ethyl acetate 8:2). Thus, 36 mg (31%) of (3′-hydroxy-8′-vinylestra-1′,3′,5′(10′)-trien-17′β-yl)-3-sulfamoyl benzoate was obtained.

¹H-NMR (CDCl₃): 0.92 (s, 3H, H-18), 4.82 (t, 1H, H-17), 6.39 (d, 1H, H-4), 6.48 (dd, 1H, H-2), 7.00 (d, 1H, H-1).

### EXAMPLE 2

### (3′-Hydroxy-8′-vinylestra-1′,3′,5′(10′)-trien-17′β-yl)-4-sulfamoyl benzoate

### ((3′-(tert-Butyldimethylsilyloxy)-8′-vinylestra-1′,3′,5′(10′)-trien-17′β-yl)-4-sulfamoyl benzoate

750 mg (2.6 mmol) of 4-sulfamido-benzoyl chloride and 28 mg of 4-dimethylaminopyridine were added to 300 mg of 3-(tert-butyldimethylsilyloxy)-8-vinylestra-1,3,5(10)-trien-17β-ol in 4 ml of pyridine and stirred at room temperature for 2 hours. The reaction mixture was poured into ice water, the precipitate was filtered off, and the thus obtained crude product (913 mg) was used without additional purification in the next stage.

### (3′-Hydroxy-8′-vinylestra-1′,3′,5′(10′)-trien-17′β-yl)-4-sulfamoyl benzoate

278 mg of tetrabutylammonium fluoride was added to 913 mg of crude ((3′-(tert-butyldimethylsilyloxy)-8′-vinylestra-1′,3′,5′(10′)-trien-17′β-yl)-4-sulfamoyl benzoate in 30 ml of tetrahydrofuran, and it was stirred at room temperature for 2 hours. Then, the reaction solution was taken up in methylene chloride and water, the organic phase was washed with water, dried on sodium sulfate and concentrated by evaporation in a vacuum. The crude product was purified by column chromatography on silica gel (cyclohexane, ethyl acetate 1:1) and recrystallized from methanol. In this way, 147 mg (42%) of (3′-hydroxy-8′-vinylestra-1′,3′,5′(10′)-trien-17′β-yl)-4-sulfamoyl benzoate was obtained.

¹H-NMR (CDCl₃): 0.96 (s, 3H, H-18), 4.87 (t, 1H, H-17), 6.51 (d, 1H, H-4), 6.60 (dd, 1H, H-2), 7.10 (d, 1H, H-1).

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius, and all parts and percentages are by weight, unless otherwise indicated.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

### REFERENCES

1. Cummings, S. R.; Browner, W. S.; Bauer, D.; Stone, K.; Ensrud, K.; Jamal, S. and Ettinger, B. (1998), Endogenous Hormones and the Risk of Hip and Vertebral Fractures Among Older Women. N. Engl. J. Med. 339, 733-38.
2. Frank, G. R. (1995), The Role of Estrogen in Pubertal Skeletal Physiology: Epiphyseal Maturation and Mineralization of the Skeleton. Acta Paediatr. 84(6), 627-30.
3. Goldzieher, J. W. (1990), Selected Aspects of the Pharmacokinetics and Metabolism of Ethinyl Estrogens and their Clinical Implications. Am. J. Obstet. Gynecol. 163, 318-22.
4. Gustafsson, J. A. (2000), Novel Aspects of Estrogen Action. J. Soc. Gynecol. Investig. 7, S8-S9.
5. Helmer, O. M., and Griffith, R. S. (1952), The Effect of the Administration of Estrogens on the Renin-Substrate (Hypertensinogen) Content on Rat Plasma. Endocrinology 51, 421-6.
6. Kelly, J. J.; Rajkovic, I. A.; O'Sullivan, A. J.; Sernia, C. and Ho, K. K. Y. (1993), Effects of Different Oral Estrogen Formulations on Insulin-like Growth Factor-I, Growth Hormone and Growth Hormone Binding Protein in Post-Menopausal Women. Clin. Endocrinol. 39, 561-67.
7. Krattenmacher, R.; Knauthe, R.; Parczyk, K.; Walker, A.; Hilgenfeldt, U. and Fritzemeier, K.-H. (1994), Estrogen Action on Hepatic Synthesis of Angiotensinogen and IGF-I: Direct and Indirect Estrogen Effects. J. Steroid. Biochem. Mol. Biol. 48, 207-14.
8. Le Roith and Butler, A. A. (1999), Insulin-like Growth Factors in Pediatric Health and Disease. J. Clin. Endocrinol. Metab. 84, 4355-61.
9. Mandel, F. P.; Geola, F. L.; Lu, J. K. H.; Eggena, P.; Sambhi, M. P.; Hershman, J. M. and Judd, H. L. (1982), Biologic Effects of Various Doses of Ethinyl Estradiol in Postmenopausal Women. Obstet. Gynecol. 59, 673-9.
10. Mashchak, C. A.; Lobo, R. A.; Dozono-Takano, R.; Eggena, P.; Nakamura, R. M.; Brenner, P. F. and Mishell, D. R., Jr. (1982), Comparison of Pharmacodynamic Properties of Various Estrogen Formulations. Am. J. Obstet. Gynecol. 144, 511-18.
11. Oelkers, W. K. H. (1996), Effects of Estrogens and Progestagens on the Renin-Aldosterone System and Blood Pressure. Steroids 61, 166-71.
12. O'Sullivan, A. J. and Ho, K. K. Y. (1995), A Comparison of the Effects of Oral and Transdermal Estrogen Replacement on Insulin Sensitivity in Postmenopausal Women. J. Clin. Endocrinol. Metab. 80, 1783-8.
13. Span, J. P. T.; Pieters, G. F. F. M.; Sweep, C. G. J.; Hermus, A. R. M. M. and Smals, A. G. H. (2000), Gender Difference in Insulin-like Growth Factor I Response to Growth Hormone (GH) Treatment in GH-Deficient Adults: Role of Sex Hormone Replacement. J. Clin. Endocrinol. Metab. 85, 1121-5.
14. von Schoultz, B.; Carlström, K.; Collste, L.; Eriksson, A.; Henriksson, P.; Pousette, A. and Stege, R. (1989), Estrogen Therapy and Liver Function—Metabolic Effects of Oral and Parenteral Administration. Prostate 14, 389-95.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding German application No. 10 2005 05 7225.1, filed Nov. 28, 2005, and U.S. Provisional Application Ser. No. 60/742,557, filed Dec. 6, 2005, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Sulfamoyl compounds of 8β-substituted estratrienes of general formula (I), 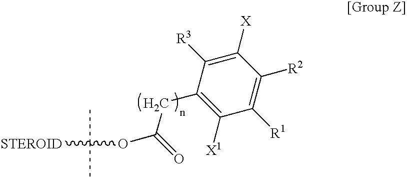
in which n can mean a number 0-4,
R¹ means a radical —SO₂NH₂ or —NHSO₂NH₂,
whereby R², R³ and X, X¹, independently of one another, stand for a hydrogen atom, a halogen atom, a nitrile group, a nitro group, a C₁₋₅-alkyl group, a CₚF₂ₚ₊₁ group with p=1-3, a group OC(O)—R²⁰, COOR²⁰, OR²⁰, C(O)NHR²⁰or OC(O)NH—R²¹,
whereby R²⁰ and R²¹ are a C₁₋₅-alkyl group, a C₃₋₈-cycloalkyl group, an aryl group, a C₁₋₄-alkylene aryl group, a C₁₋₄-alkylene-C₃₋₈-cycloalkyl group or a C₃₋₈-cycloalkylene-C₁₋₄-alkyl group, and
R²⁰ in addition can mean a hydrogen atom, or
R² can mean a radical —SO₂NH₂ or —NHSO₂NH₂,
whereby R¹, R³ and X, X¹, independently of one another, stand for a hydrogen atom, a halogen atom, a nitrile group, a nitro group, a C₁₋₅-alkyl group, a CₚF₂ₚ₊₁ group with p=1-3, a group OC(O)—R²⁰, COOR²⁰, OR²⁰, C(O)NHR²⁰ or OC(O)NH—R²¹,
whereby R²⁰ and R²¹ are a C₁₋₅-alkyl group, a C₃₋₈-cycloalkyl group, an aryl group, a C₁₋₄-alkylene aryl group, a C₁₋₄-alkylene-C₃₋₈-cycloalkyl group or C₃₋₈-cycloalkylene-C₁₋₄-alkyl group, and
R²⁰ in addition can mean a hydrogen, or
R³ can mean a radical —SO₂NH₂ or —NHSO₂NH₂,
whereby R¹, R² and X, X¹, independently of one another, stand for a hydrogen atom, a halogen atom, a nitrile group, a nitro group, a C₁₋₅-alkyl group, a CₚF₂ₚ₊₁ group with p=1-3, a group OC(O)—R²⁰, COOR²⁰, OR²⁰, C(O)NHR²⁰ or OC(O)NH—R²¹,
whereby R²⁰ and R²¹ are a C₁₋₅-alkyl group, a C₃₋₈-cycloalkyl group, an aryl group, a C₁₋₄-alkylene aryl group, a C₁₋₄-alkylene-C₃₋₈-cycloalkyl group or C₃₋₈-cycloalkylene-C₁₋₄-alkyl group, and
R²⁰ in addition can mean a hydrogen, and
STEROID stands for a steroidal ABCD-ring system of formula (A): 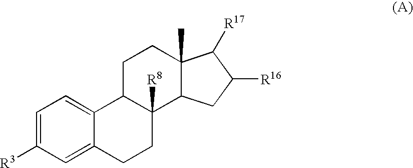
whereby the radicals R³, R⁸, R¹⁶ and R¹⁷ have the following meaning:
R³ can be Z and
R¹⁷ can be an OH group, a tri(C₁-C₄-alkyl)silyloxy group or a group OC(O)—R²⁰ or
R³ can be OH, OMe, a tri(C₁-C₄-alkyl)silyloxy group, or a group OC(O)—R²⁰ and
R¹⁷ can be Z
and
R⁸ can be a branched or straight-chain, optionally partially or completely halogenated alkyl, alkenyl or alkinyl radical with up to 3 carbon atoms,
R¹⁶ can be a hydrogen atom, a halogen atom, or a methyl group,
whereby the substituents R¹⁶ and R¹⁷ in each case can be both in α- and in β-position, and their pharmaceutically acceptable salts.

2. Compounds according to claim 1, characterized in that n is 0, 1 or 2.

3. Compounds according to claim 1, wherein in each case a radical
R¹, R² or R³ represents a group —SO₂NH₂.

4. Compounds according to claim 1, wherein R¹ represents a group
—SO₂NH₂ or —NHSO₂NH₂.

5. Compounds according to claim 4, wherein R¹ represents a group —SO₂NH₂.

6. Compounds according to claim 1, wherein if one of the radicals R¹, R², and R³ does not mean —SO₂NH₂ or —NHSO₂NH₂, the other two radicals R¹, R², and R³ in each case, as well as X and X¹, independently of one another, stand for a hydrogen, fluorine or chlorine atom, a hydroxy group or a methoxy group.

7. Compounds according to claim 1, wherein R⁸ is a methyl, ethyl, vinyl or difluorovinyl radical.

8. Compounds according to claim 1,
1) (3′-Hydroxy-8′β-methyl-estra-1′,3′,5′(10′)-trien-17′β-yl)3-sulfamoyl benzoate
2) (3′-Hydroxy-8′β-ethyl-estra-1′,3′,5′(10′)-trien-17′β-yl)3-sulfamoyl benzoate,
3) (3′-Hydroxy-8′β-vinyl-estra-1′,3′,5′(10′)-trien-17′β-yl)3-sulfamoyl benzoate,
4) (3′-Hydroxy-8′β-methyl-estra-1′,3′,5′(10′)-trien-17′β-yl)4-sulfamoyl benzoate,
5) (3′-Hydroxy-8′β-ethyl-estra-1′,3′,5′(10′)-trien-17′β-yl)4-sulfamoyl benzoate,
6) (3′-Hydroxy-8′β-vinyl-estra-1′,3′,5′(10′)-trien-17′β-yl)4-sulfamoyl benzoate
7) (3′-Acetoxy-8′β-methyl-estra-1′,3′,5′(10′)-trien-17′β-yl)3-sulfamoyl benzoate,
8) (3′-Acetoxy-8′β-ethyl-estra-1′,3′,5′(10′)-trien-17′β-yl)3-sulfamoyl benzoate,
9) (3′-Acetoxy-8′β-vinyl-estra-1′,3′,5′(10′)-trien-17′β-yl)3-sulfamoyl benzoate,
10) (3′-Acetoxy-8′β-methyl-estra-1′,3′,5′(10′)-trien-17′β-yl)4-sulfamoyl benzoate,
11) (3′-Acetoxy-8′β-ethyl-estra-1′,3′,5′(10′)-trien-17′β-yl)4-sulfamoyl benzoate,
12) (3′-Acetoxy-8′β-vinyl-estra-1′,3′,5′(10′)-trien-17′β-yl)4-sulfamoyl benzoate,
13) (3′-Benzoyloxy-8′β-methyl-estra-1′,3′,5′(10′)-trien-17′β-yl)3-sulfamoyl benzoate,
14) (3′-Benzoyloxy-8′β-methyl-estra-1′,3′,5′(10′)-trien-17′β-yl)4-sulfamoyl benzoate,
15) (3′-Benzoyloxy-8′β-ethyl-estra-1′,3′,5′(10′)-trien-17′β-yl)3-sulfamoyl benzoate,
16) (3′-Benzoyloxy-8′β-ethyl-estra-1′,3′,5′(10′)-trien-17′β-yl)4-sulfamoyl benzoate,
17) (3′-Benzoyloxy-8′β-vinyl-estra-1′,3′,5′(10′)-trien-17′β-yl)3-sulfamoyl benzoate,
18) (3′-Benzoyloxy-8′β-vinyl-estra-1′,3′,5′(10′)-trien-17′β-yl)4-sulfamoyl benzoate,
19) (3′-Hydroxy-8′β-vinyl-estra-1′,3′,5′(10′)-trien-17′β-yl)2-chloro-5-sulfamoyl benzoate,
20) (3′-Hydroxy-8′β-vinyl-estra-1′,3′,5′(10′)-trien-17′β-yl)3-sulfamoyl-4-chloro-benzoate,
21) (3′-Hydroxy-8′β-methyl-estra-1′,3′,5′(10′)-trien-17′β-yl)2-chloro-5-sulfamoyl benzoate,
22) (3′-Hydroxy-8′β-methyl-estra-1′,3′,5′(10′)-trien-17′β-yl)3-sulfamoyl-4-chlorobenzoate,
23) (3′-Hydroxy-8′β-ethyl-estra-1′,3′,5′(10′)-trien-17′β-yl)2-chloro-5-sulfamoyl benzoate,
24) (3′-Hydroxy-8′β-ethyl-estra-1′,3′,5′(10′)-trien-17′β-yl)3-sulfamoyl-4-chloro-benzoate,
25) (17′β-(n-Pentanoyloxy)-8′β-vinyl-estra-1′,3′,5′(10′)-trien-3′-yl)3-sulfamoyl benzoate,
26) (17′β-(n-Pentanoyloxy)-8′β-methyl-estra-1′,3′,5′(10′)-trien-3′-yl)3-sulfamoyl benzoate,
27) (17′β-(n-Pentanoyloxy)-8′β-ethyl-estra-1′,3′,5′(10′)-trien-3′-yl)3-sulfamoyl benzoate,
28) (17′β-Benzoyloxy-8′β-vinyl-estra-1′,3′,5′(10′)-trien-3′-yl)3-sulfamoyl benzoate,
29) (17′β-Benzoyloxy-8′β-methyl-estra-1′,3′,5′(10′)-trien-3′-yl)3-sulfamoyl benzoate,
30) (17′β-Benzoyloxy-8′β-ethyl-estra-1′,3′,5′(10′)-trien-3′-yl)3-sulfamoyl benzoate,
31) (17′β-(n-Pentanoyloxy)-8′β-vinyl-estra-1′,3′,5′(10′)-trien-3′-yl)4-sulfamoyl benzoate,
32) (17′β-(n-Pentanoyloxy)-8′β-methyl-estra-1′,3′,5′(10′)-trien-3′-yl)4-sulfamoyl benzoate,
33) (17′β-(n-Pentanoyloxy)-8′β-ethyl-estra-1′,3′,5′(10′)-trien-3′-yl)4-sulfamoyl benzoate,
34) (17′β-Benzoyloxy-8′β-vinyl-estra-1′,3′,5′(10′)-trien-3′-yl)4-sulfamoyl benzoate,
35) (17′β-Benzoyloxy-8′β-methyl-estra-1′,3′,5′(10′)-trien-3′-yl)4-sulfamoyl benzoate,
36) (17′β-Benzoyloxy-8′β-ethyl-estra-1′,3′,5′(10′)-trien-3′-yl)4-sulfamoyl benzoate,
37) (17′β-Acetoxy-8′β-vinyl-estra-1′,3′,5′(10′)-trien-3′-yl)3-sulfamoyl benzoate,
38) (17′β-Acetoxy-8′β-ethyl-estra-1′,3′,5′(10′)-trien-3′-yl)3-sulfamoyl benzoate,
39) (17′β-Acetoxy-8′β-methyl-estra-1′,3′,5′(10′)-trien-3′-yl)3-sulfamoyl benzoate,
40) (17′β-Acetoxy-8′β-vinyl-estra-1′,3′,5′(10′)-trien-3′-yl)4-sulfamoyl benzoate
41) (17′β-Acetoxy-8′β-ethyl-estra-1′,3′,5′(10′)-trien-3′-yl)4-sulfamoyl benzoate,
42) (17′β-Acetoxy-8′β-methyl-estra-1′,3′,5′(10′)-trien-3′-yl)4-sulfamoyl benzoate,
43) (3′-Methoxy-8′β-vinyl-estra-1′,3′,5′(10′)-trien-17′β-yl)3-sulfamoyl benzoate,
44) (3′-Methoxy-8′β-ethyl-estra-1′,3′,5′(10′)-trien-17′β-yl)3-sulfamoyl benzoate,
45) (3′-Methoxy-8′β-methyl-estra-1′,3′,5′(10′)-trien-17′β-yl)3-sulfamoyl benzoate,
46) (3′-Methoxy-8′β-vinyl-estra-1′,3′,5′(10′)-trien-17′β-yl)4-sulfamoyl benzoate,
47) (3′-Methoxy-8′β-ethyl-estra-1′,3′,5′(10′)-trien-17′β-yl)4-sulfamoyl benzoate,
48) (3′-Methoxy-8′β-methyl-estra-1′,3′,5′(10′)-trien-17′β-yl)4-sulfamoyl benzoate.

9. Pharmaceutical compositions that contain at least one compound according to claim 1 as well as a pharmaceutically compatible vehicle.

10. Pharmaceutical composition according to claim 9, wherein at least one additional steroidal compound is included.

11. Pharmaceutical composition according to claim 10, wherein the additional steroidal compound is a gestagen, an antigestagen or a mesoprogestin.

12. Pharmaceutical composition according to claim 11, wherein the gestagen is drospirenone, dienogest, norethisterone or levonorgestrel, the antigestagen is onapristone or mifepristone or the mesoprogestin is asoprisnil.

13. Use of the compounds according to the invention according to claim 1 for the production of a pharmaceutical agent.

14. Use according to claim 13 for treating diseases and conditions in women and in men that are caused by an estrogen deficiency.

15. Use according to claim 13 for treating perimenopausal and postmenopausal symptoms.

16. Use according to claim 13 for the in-vitro treatment of male infertility.

17. Use according to claim 13 for the in-vivo treatment of male infertility.

18. Use according to claim 13 for the in-vitro treatment of female infertility.

19. Use according to claim 13 for the in-vivo treatment of female infertility.

20. Use according to claim 13 for the therapy of hormone-deficiency-induced symptoms in ovarian dysfunction that is caused by surgery, medication, etc.

21. Use according to claim 13 for hormone replacement therapy (HRT).

22. Use according to claim 20 in combination with a selective estrogen receptor modulator (SERM), for example raloxifene.

23. Use according to claim 13 for prophylaxis and therapy of a hormone-deficiency-induced bone mass loss.

24. Use according to claim 13 for prophylaxis and therapy of osteoporosis.

25. Use according to claim 23 in combination with the natural vitamin D3 or with calcitriol analogs for bone building or as supporting therapies to therapies that cause a bone mass loss (for example, a therapy with glucocorticoids, aromatase inhibitors, GnRH agonists or antagonists, or chemotherapy).

26. Use according to claim 13 for prevention against and therapy of cardiovascular diseases.

27. Use according to claim 13 for treating inflammatory diseases and diseases of the immune system.

28. Use according to claim 27 for treating rheumatoid arthritis.

29. Use according to claim 27 for treating multiple sclerosis, Crohn's disease or endometriosis.

30. Use according to claim 13 for preventing and treating benign prostate hyperplasia (BPH).

31. Use according to claim 30 in combination with antiestrogens and selective estrogen receptor modulators for preventing and treating benign prostate hyperplasia (BPH).

32. Use according to claim 31, whereby 7alpha-[9-[(4,4,5,5,5-pentafluoropentyl)sulfinyl]nonyl]estra-1,3,5(10)-triene-3,17β-diol (fulvestrant) is used as antiestrogen or raloxifene, tamoxifen, or 5-(4-{5-[(RS)-(4,4,5,5,5-pentafluoropentyl)sulfinyl]-pentyl}phenyl)-6-phenyl-8,9-dihydro-7H-benzocyclohepten-2-ol is used as SERM.

33. Use according to claim 13 for treating arthritic symptoms, in particular after therapies that result in estrogen deprivation, for example after treatment with aromatase inhibitors or GnRH antagonists or agonists.

34. Use of compounds according to claim 1 for the production of pharmaceutical agents for treating diseases that can be influenced positively by the inhibition of the carbonic anhydrase activity.

35. Use of compounds according to claim 1 for the production of pharmaceutical agents for treating alopecia.

36. Use of 3′-hydroxy-8′β-vinyl-estra-1′,3′,5′(10′)-trien-17′β-yl)3-sulfamoyl benzoate, (3′-hydroxy-8′β-vinyl-estra-1′,3′,5′(10′)-trien-17′β-yl)4-sulfamoyl benzoate, 3′-hydroxy-8′β-vinyl-estra-1′,3′,5′(10′)-trien-17′β-yl)3-sulfamoyl-4-chlorobenzoate, (3′-methoxy-8′β-vinyl-estra-1′,3′,5′(10′)-trien-17′β-yl)3-sulfamoyl benzoate and (3′-methoxy-8′β-vinyl-estra-1′,3′,5′(10′)-trien-17′β-yl)4-sulfamoyl benzoate according to claim 13.

37. Process for the production of compounds of general formula (I) according to claim 1 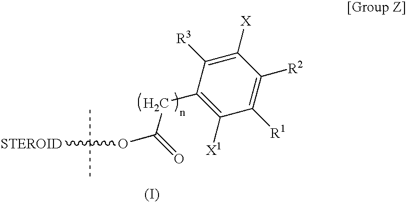
by reaction of 8β-substituted estratrienes according to formula (A) with corresponding sulfamoylphenylcarboxylic acid or derivatives thereof or by reaction of corresponding compounds with sulfamide, sulfamoyl chloride or aminosulfonyl isocyanate.
